(19) United States
(12) Patent Application Publication
(10) Pub. No.: US 2013/0144249 A1
(43) Pub. Date: Jun. 6, 2013

(54) ADHESIVE DELIVERY DEVICES, SYSTEMS AND METHODS

(71) Applicants: Paul V. Fenton, Marblehead, MA (US); J. Christopher Flaherty, Topsfield, MA (US); Paul A. Westhaver, Dartmouth (CA); Anshuman Shrivastava, Adhartal (IN)

(72) Inventors: Paul V. Fenton, Marblehead, MA (US); J. Christopher Flaherty, Topsfield, MA (US); Paul A. Westhaver, Dartmouth (CA); Anshuman Shrivastava, Adhartal (IN)

(73) Assignee: DARTMOUTH MEDICAL RESEARCH LIMITED, Halifax, NS (CA)

(21) Appl. No.: 13/512,726

(22) PCT Filed: Nov. 30, 2010
(86) PCT No.: PCT/US10/03060

§ 371 Date: Feb. 15, 2013

Related U.S. Application Data
(60) Provisional application No. 61/265,267, filed on Nov. 30, 2009.

(51) Int. Cl. A61B 17/00; A61M 5/19

(52) U.S. Cl. CPC A61B 17/00491; A61M 5/19
USPC 604/500; 604/239; 604/82; 604/113

(57) ABSTRACT

Devices, systems and methods are disclosed for delivering adhesive material to a patient site. A delivery device includes a housing and a nozzle. Adhesive material exits the end of the nozzle such as to adhesively attach tissue to tissue or a material or device to tissue.

### BACKGROUND OF THE INVENTION

The present invention relates generally to adhesive delivery devices, systems and methods. In particular, the present invention provides a transcutaneous adhesive delivery device which provides simplified injectable fixation and repair of tissues especially bone tissue.

Numerous devices have been used to repair bone fractures. Plates, pins and screws along with other implantable devices are common devices used in the repair of bone fractures. Plates and reinforcing mechanical fixtures are provided in various types, and are used in combination with pins, screws and other attachment means to repair bone tissue. These mechanical devices are used to mechanically fixate the bone tissue to provide stabilization and fixation to improve the healing process. Many of the mechanical bone fixation devices and methods are dependent on technique and can result in inadequate attachment, excessive time to install, undesirable long term effects of permanent implants such as infection, rejection, scarring and pain. These mechanical fixation devices also require surgery to implant. There is therefore a need for improved bone repair fixation devices, materials, systems and methods.

### SUMMARY OF THE INVENTION

Several unique bone repair and soft tissue repair adhesive systems and tissue adhesion methods are provided which provide simplified, repeatable and reliable fixation of bone tissue and soft tissue to one or more structures.

According to a first aspect of the invention, a transcutaneous fixation system for repairing fractured bone tissue of a patient is provided. The adhesive is a flowable polymer that is formulated to hold bone fragments together during the healing process. A delivery device comprises housing with a proximal end and a distal end; and an elongate penetrating portion comprising a proximal end, a distal end and exit means. The penetrating portion is configured to penetrate tissue. The penetrating portion has an interior passage to allow for the flowable repair material to move from the housing through the proximal end and exit the distal end. The penetrating portion includes exit means configured to allow a controlled flow to pass out into the site of the repair.

In a preferred embodiment, the adhesive material may be bioabsorbable, bioerodible or biodegradable, hereinafter “bioabsorbable”, or the material may include bioabsorbable materials. The flow of the material may require heating to make the material achieve a flowable state, or the material may be configured to flow at room temperature.

In another preferred embodiment, the delivery device housing is fixedly attached to the penetrating portion. In yet another preferred embodiment, one or more portions of the delivery device can be controllably changed in rigidity or shape. In a particular embodiment, the penetrating portion is selectively made rigid or flexible to assist in tissue penetration and manipulation within the tissue to reach the desired site of repair.

In yet another preferred embodiment, the delivery device housing has a receptacle for adhesive material that allows for transport from its proximal end to its distal end and into the proximal end of the penetrating portion. The device is configured such that advancing the adhesive material through the penetrating portion results in a flow and dispensing at the distal to repair the selected tissue. The advancement of the material is controlled by means of an actuator engaged by the surgeon or operator. Retraction of the delivery device can be accomplished by manual or automated means.

In still yet another preferred embodiment, the material that constitutes the adhesive may be non-absorbable or may contain structures, either spheres or other geometric shapes, solid or hollow, that may be made from materials with a wide range of properties including, among others, melting temperature and absorbability.

### BRIEF DESCRIPTION OF THE DRAWINGS

The accompanying drawings, which are incorporated in and constitute a part of this specification, illustrate various embodiments of the present invention, and, together with the description, serve to explain the principles of the invention. In the drawings:

FIG. 1 illustrates a side view of a transcutaneous adhesive fixation system penetrating soft tissue to the site of a bone fracture, consistent with the present invention.

FIG. 2 illustrates a side view of the device of FIG. 1 being used to repair a bone, consistent with the present invention.

FIG. 3 illustrates multiple side sectional views of a transcutaneous adhesive fixation system including an inner member and being used to treat bone tissue, consistent with the present invention.

FIG. 4 illustrates a perspective view of the device of FIG. 1 being used with an implantable mesh to treat tissue, consistent with the present invention.

FIG. 5 illustrates a side view of the device of FIG. 1 being used to attach an implant to bone, consistent with the present invention.

FIG. 6 illustrates a side view of a transcutaneous adhesive fixation system including markings on the elongated portions, consistent with the present invention.

FIG. 7 illustrates views of a microsphere encapsulation and an adhesive material including hollow bodies, consistent with the present invention.

FIG. 8 illustrates a perspective view of the device of FIG. 1 being use to fabricate implants, consistent with the present invention

FIG. 9 illustrates a side, cutaway view of the distal, penetrating tip portion of the device of FIG. 1, consistent with the present invention.

FIG. 10 illustrates side and side cutaway views of the penetrating portion of the device of FIG. 1, consistent with the present invention.

FIG. 11 illustrates a side sectional view of an implant placed into bone and fixated with a transcutaneous fixation device, consistent with the present invention.

### DESCRIPTION OF THE EMBODIMENTS

Reference will now be made in detail to the present embodiments of the invention, examples of which are illustrated in the accompanying drawings. Wherever possible, the same reference numbers will be used throughout the drawings to refer to the same or like parts.

The present invention provides devices, systems and methods for repair, and fixation of bone and other tissues of a patient using adhesives. Repair and fixation of bone tissue is used in many medical procedures, including trauma, implant surgery, reconstructive surgery, and other procedures which cause the need for a repair. Numerous other types of tissue may also require fixation suited for this system such as but not limited to ligaments, tendon, muscle, cartilage, and skin.

Definitions: To facilitate an understanding of the invention, a number of terms are defined below.

As used herein, the terms “subject” and “patient” refer to any animal, such as a mammal like livestock, pets, and preferably a human. Specific examples of “subjects” and “patients” include, but are not limited, to individuals requiring medical assistance, and in particular, requiring tissue fixation.

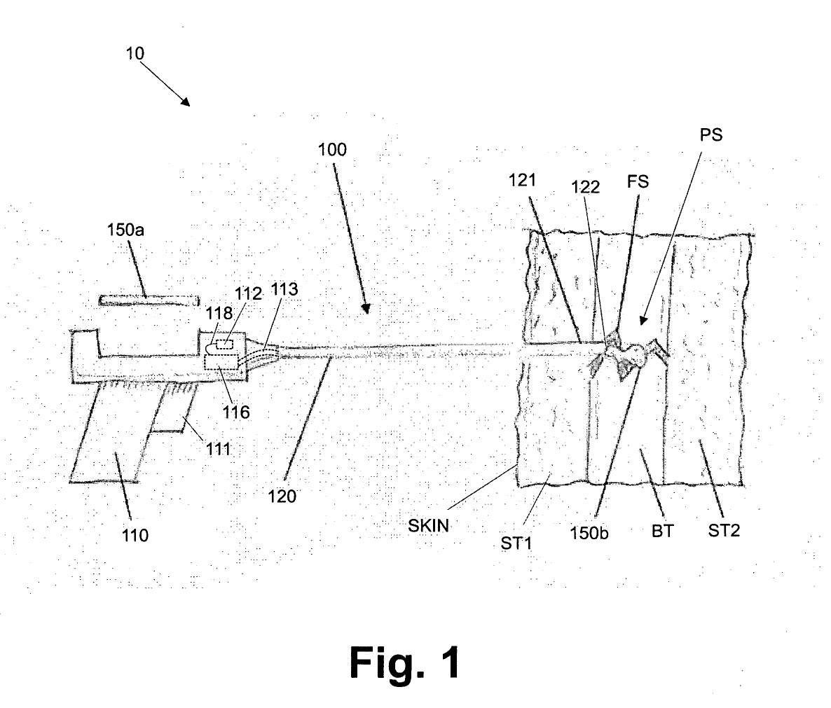

The present invention provides structures that embody aspects of a bone repair system and numerous other tissue repair and fixation systems. The present invention also provides a delivery device for adhesive material. The present invention also provides a flowable adhesive material, such as for attaching tissue to tissue or for fixating or otherwise implanting devices in a patient. The device and system of the present invention can be used to attach one or more tissue or artificial structure to each other. The illustrated and preferred embodiments discuss these structures and techniques in the context of tissue fixation. These structures, systems, and techniques are well suited for use in the field of surgery and other medical procedures. However, it should be appreciated that the invention is applicable for use in other applications that affix a first structure to a second structure at a patient site. The fixation devices, systems and method of the present invention have advantages over previous prior art devices. FIGS. 1-11 show various preferred embodiments of the devices and systems of the present invention. The present invention is not limited to these particular configurations.

Referring now to FIG. 1, a preferred embodiment of an adhesive material injection system of the present invention is illustrated. The adhesive material injection system is configured to be used with one or more delivery devices and one or more adhesive materials to allow a clinician such as a surgeon, to implant or otherwise deploy an adhesive material into or onto bone or other tissue of a patient, such as to make a bone repair. System 10 includes adhesive injection device 100, which includes a housing 110, of a pistol grip construction, and nozzle 120, an elongate tube with one or more lumens, not shown but traveling from the proximal end to the distal end of nozzle 120. Nozzle 120 may be removably attached to housing 110 at attachment means 113, preferably a threaded assembly which mates with threads on the proximal end of nozzle 120, threads not shown or with a snap fit assembly. Nozzle 120 has have a penetrating portion, distal portion 121 which includes sharpened distal tip 122 shown as having been advanced through the patient's skin to patient site PS. In an alternative embodiment, distal portion 121 is not configured to penetrate tissue but rather avoid tissue tearing, such as when distal tip 122 has an atraumatic edge. Nozzle 120 is of sufficient length to reach the intended patient sites for the delivery of adhesive material, such as via one or more transcutaneous and/or transosseous routes. In an alternative embodiment, nozzle 120 has a telescoping construction, such as an advanceable telescope that can be locked in one or more different overall lengths.

Adhesive material 150a, typically a bone adhesion material, is shown exterior to housing 110, such as to be placed in a solid state form in reservoir 114 to refill device 100 with additional adhesive material. Reservoir is in fluid communication with heater unit 116 and nozzle 120 respectively. Heater unit 116 is configured to apply heat to adhesive material 150a, such as to a temperature slightly above body temperature, to allow it to flow through nozzle 120 to a location proximate patient site PS. Heater unit 116 maintains adhesive material 150a in a flowable state, such as a state that can be molded and/or has a reduced viscosity. Heater unit 116 is attached to power supply 112, preferably a battery, such that when trigger 111 of housing 110 is activated, adhesive material 150a flow through nozzle 120. Heater unit 116 may include one or more temperature sensors, not shown, but preferably thermocouples or thermistors configured to maintain adhesive material 150a at a specific temperature or within a specific temperature range.

As shown in FIG. 1, distal portion 121 has penetrated through soft tissue ST1 and into bone tissue BT, at fracture site FS. Adhesive material 150b, which may be in a solid or other non-flowable state after having cooled to body temperature.

Adhesive material 150a is a formulation of one or more biocompatible materials such as polymers. Adhesive material 150a may be made of materials which will remain intact, permanently implanted over long periods of time, such as times greater than 6 months. Alternatively, adhesive material 150a may be made of materials which bioabsorb, such as at a bioabsorption rate of less than six months, less than 1 month, or even less than seven days. Numerous materials have been developed to be absorbed by the body, such as a magnesium reinforced polymer. Numerous polymers can be used such as polymers selected from the group consisting of: polylactide, poylglycolide, polysaccharides, proteins, polyesters, polyhydroxyal kanoates, polyalkelene esters, polyamides, polycaprolactone, polyvinyl esters, polyamide esters, polyvinyl alcohols, polyanhydrides and their copolymers, modified derivatives of caprolactone polymers, polytrimethylene carbonate, polyacrylates, polyethylene glycol, polyolefin, engineered materials, hydrogels, photo-curable hydrogels, terminal diols, minerals, and combinations of these. Bioabsorbable fibers that reinforce a bioabsorbable polymer matrix can be used. Materials can be made in permanent or absorbable matrices and can include minerals and therapeutics as one of the constituents.

In an alternative embodiment, the adhesive material 150 includes two separate substances. The substances may be mixed prior to placing in device 100, may be mixed within device 100, or they may be delivered separately to the patient site, simultaneously and/or sequentially. The two separate substances may have different bioabsorption rates, different long term rigidity, or other different pre or post dispensing properties. In one embodiment, adhesive material 150 includes three or more different substances. In an alternative embodiment, adhesive material 150a is combined with a permanent or absorbable portion, such as a portion including a filament loop such as filament loop 152 of FIG. 3. In the configuration of FIG. 3, the adhesive material of the present invention and an anchor, filament loop 152 are used to create an anchor-like deployment in a bone structure.

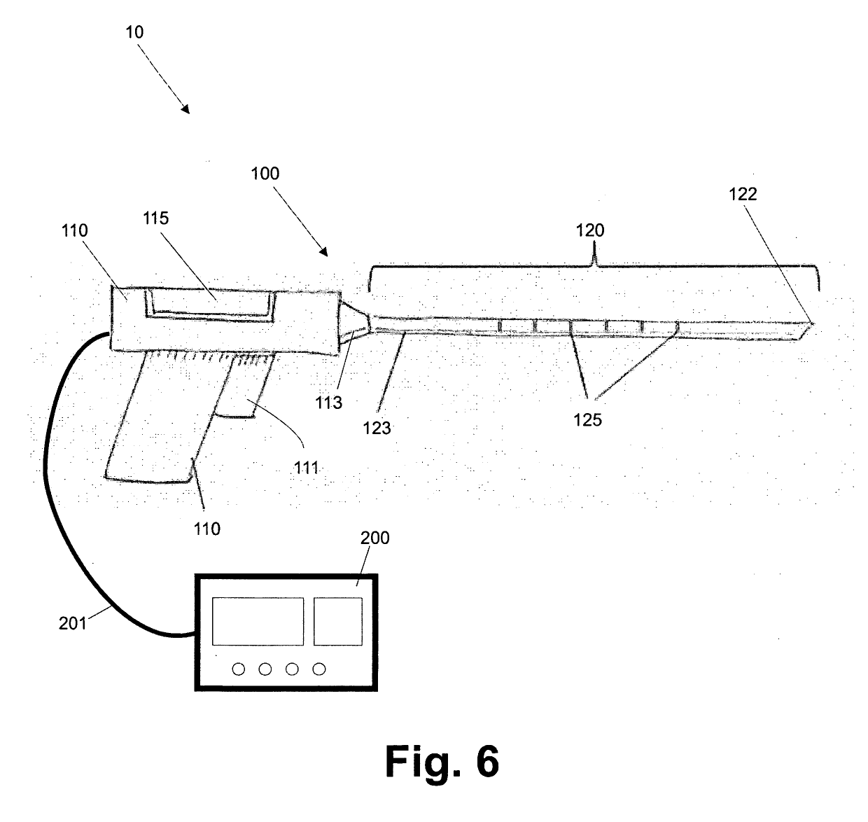

System 10 and/or delivery device 100 can be configured to operate in a manual or an automated mode. Adhesive material 150a can be delivered via manual pumping mechanisms or automated, pressurized or otherwise powered delivery. Pumping means may be included in housing 110, or a separate device as is described in reference to FIG. 6 here below. Adhesive material can be dispensed in predetermined amounts (e.g. a predetermined volume with each depression of the trigger), or continuously dispensed through nozzle 120 as long as the trigger is activated.

The patient sites of the present invention may include numerous forms of tissue including soft tissues such as cartilage and ligaments and hard tissue such as bone. In one embodiment, hard tissue for application of the adhesive material is selected from the group consisting of: surgically cut bone such as cut sternum from a open heart procedure; complex bone fractures such as bone fractures difficult to address with pins and/or screws; bone defects such as a bone defect to be filled with the adhesive material. In another embodiment, soft tissue from the knee is repaired with the system 10 such as to repair “bucket handle” tears or radial tears of the menisci. In yet another embodiment, system 10 is used in a spinal procedure, such as to repair a cervical disc after a nucleotomy procedure. In yet another embodiment, system 10 is used in a cardiac procedure, such as to repair a heart defect, such as an opening in the foramen ovale, or to fill the left atrial appendage, such as to reduce the likelihood of clot formation in a patient with a cardiac arrhythmia. In yet another embodiment, system 10 is used to treat a vascular defect, such as an aneurysm. In yet another embodiment, system 10 is used in a lung procedure, such as to repair one or more leaks in lung tissue. In yet another embodiment, system 10 is used in a procedure in the nasal cavity. In yet another embodiment, system 10 is used to close a surgical incision or to stop bleeding. In yet another embodiment, system 10 may be used to repair a fractured clavicle or associated tendons, such as to affix bone to bone and/or to adhere soft tissue segments to the receiving bone section.

A first tissue portion may be attached to a second tissue portion, where the first tissue portion and the second tissue portion have similar or dissimilar characteristics. The system of the present invention can be used to attach soft tissue to bone, bone to bone, and soft tissue to soft tissue. In addition to the treatment of bone defects by filling one or more voids, the system of the present invention can be used to fill other void areas such as the area vacated by a resected tumor. In addition, the adhesive material of the present invention may be used to modify (e.g. cover with a smooth adhesive material surface) a sharp or otherwise traumatic surface, such as a bone spur; a broken bone; a bone chip; a bone screw head or other implanted screw head; and combinations of these.

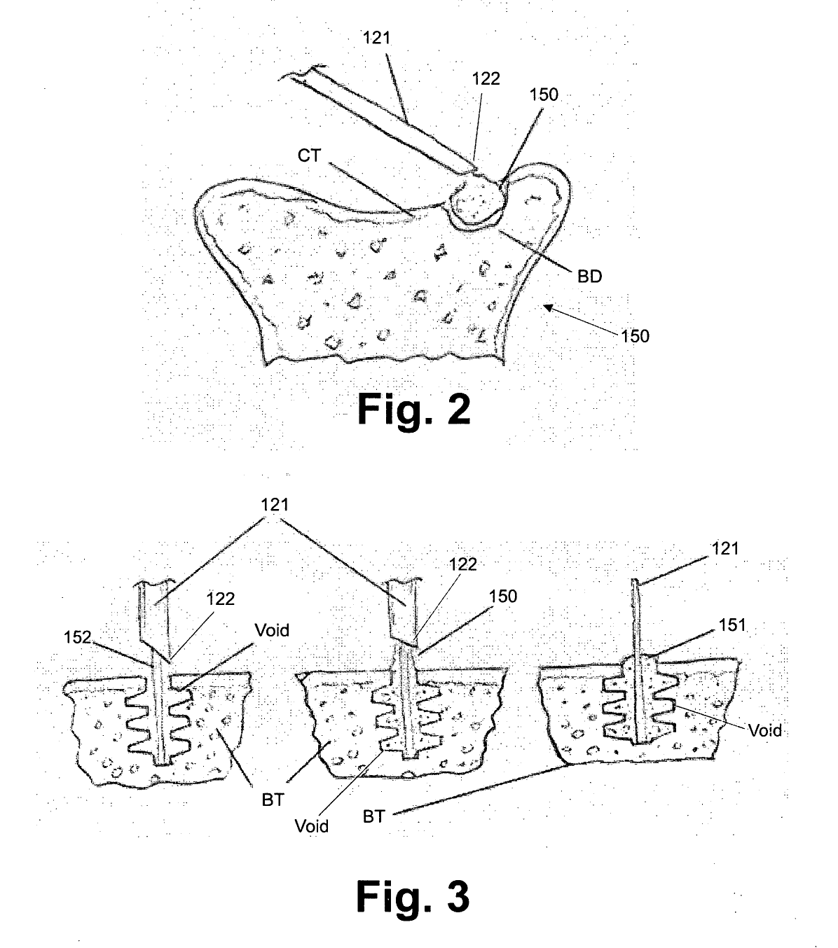

Referring now to FIG. 2, a preferred method of using device 100 of FIG. 1 is illustrated. Bone B includes bone defect BD where cartilage tissue CT and the underlying bone structure has eroded. Distal portion 121 of the device of the present invention is being used to fill bone defect BD with adhesive material 150, such that a spherical mass of adhesive 150 is formed at tip 122 and into bone defect BD.

Referring now to FIG. 3, three cross sections of bone tissue are shown in which a void V has been created in each bone tissue BT by a clinician such as a surgeon to receive the adhesive material of the present invention. The adhesive material is provided via the delivery device of the present invention through distal portion 121 and tip 122 to produce a fixation point for attachment of a second tissue or structure the patient site. This embodiment could be used for attachment of a soft tissue like a ligament or tendon to the bone as in rotator cuff repair of the shoulder joint. In the far left drawing, a filament loop 152 is being delivered through tip 122 and into the void V. In a subsequent step, adhesive material would be delivered to permanently or temporarily fix the filament material to the void V. In the middle drawing of FIG. 3, adhesive material 150 is in a flowable state. In the far right drawing of FIG. 3, the adhesive material 151 is in a solid or other non-flowable state.

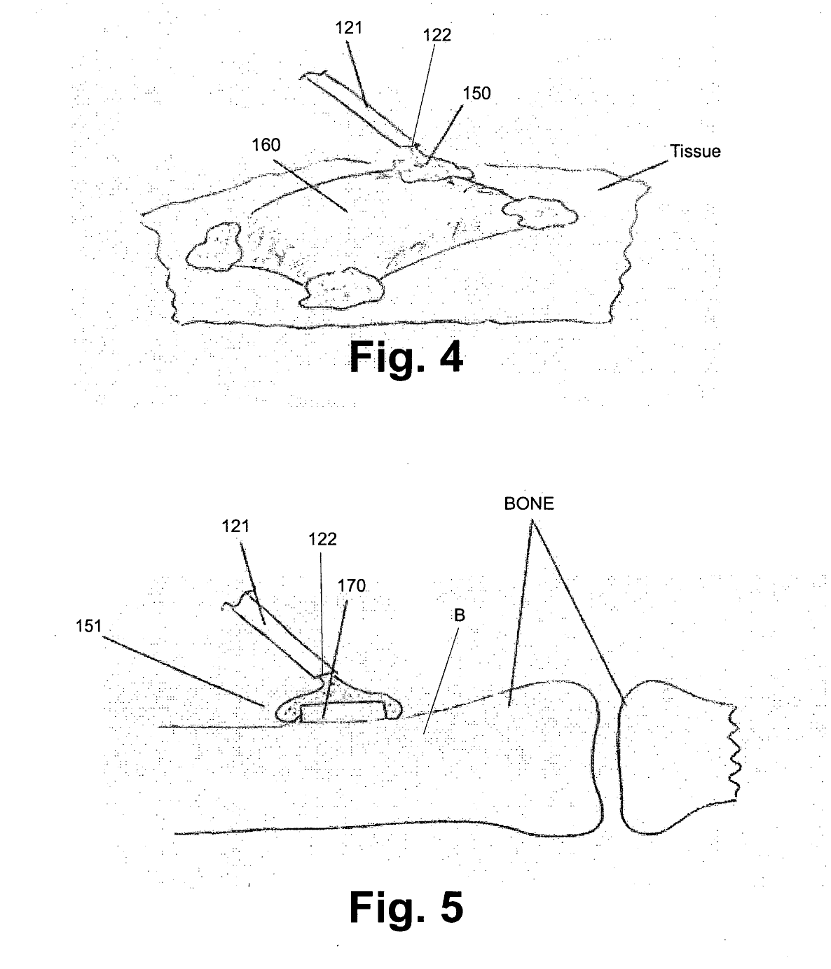

Referring now to FIG. 4, a perspective view of a preferred system and method of the present invention is illustrated where a membrane is fixed to a patient site with the adhesive and device of the present invention. Mesh fabric 160 can be used to create a support structure such as in a hernia repair procedure, shoulder repair procedure, skin repair such as in attaching a skin graft in burned or lacerated skin repair, or other procedure in which an additional support is beneficial. Numerous biocompatible mesh materials can be used such as Dacron mesh. Mesh 160 can be placed within the body or on the surface of the skin or tissue surface such as in a procedure treating one or more patient burns or other tissue surface repairs. Adhesive material 150 is shown being delivered at four corners of mesh 160 which has been placed on the surface of the patient's tissue. Material 150 is delivered through distal portion 121 through tip 122.

Referring now to FIG. 5, a perspective view of a preferred system and method of the present invention is illustrated where an implantable device is fixed to a patient site with the adhesive and device of the present invention. A bone joint is shown onto which an implant 170 is adhered to the surface by flowable material 150 via distal portion 121 and tip 122. In a preferred embodiment, the bone is the skull of the patient, such as when an implant is fixed to a recess made in the skull to accommodate the implant, such as a cochlear implant, brain implant, and/or transceiver device for communication with another implanted device. The system of the present invention may also be used to repair a craniotomy, such as in fixing the removed skull portion to the craniotomy site. In an alternative embodiment, flowable material 150 is used to attach a substance or a device to tissue, such as a gel or a foam or a microchip.

Referring now to FIG. 6, a side view of a system of the present invention is illustrated. Delivery device 100 includes an elongate tube, nozzle 120 which includes at least one lumen there through, not shown but in fluid communication with one or more internal components of housing 110 and sized to allow adhesive material, in a flowable state, to flow through the lumen. Nozzle 120 is preferably rigid, but may be flexible or include flexible portions such as hinged rigid portions. Nozzle 120 may be configured to transition from rigid to flexible or vice versa, such as via a mechanism, not shown but preferably selected from the group consisting of: hydraulic or pneumatic chambers, embedded shaped memory material, insertable pre-shaped mandrels and combinations of these. Nozzle 120 may be malleable, such as via the inclusion of one or more plastically deformable wires or rods. In an alternative embodiment, distal portion 120 comprises a rolled sheet of material, such as rolled Nitinol or stainless steel sheet, and a through lumen is formed or otherwise increased in diameter, by unfurling (unrolling) the sheet.

Nozzle 120 may include one or more markers 126. Markers 126 are preferably markers selected form the group consisting of: visible and non-visible markers; ultrasonically reflective markers; radiopaque markers; magnetic markers; electromagnetic markers; and combinations of these. These markers may be used to determine an insertion depth (e.g. into tissue) and/or otherwise orient device 100 for tissue fixation, bone repair, material delivery, sealing, or other procedures requiring adhesive delivery.

Nozzle 120 may have a circular cross-section, such as when the nozzle is a round tube, or alternative geometries may be employed. Alternative geometries include but are not limited to: oval, square, rectangular and trapezoidal, such as to create a preferred bending moment of nozzle 120, for preferred insertion of one or more devices into nozzle 120, such as one or more stiffening devices such as straight or curved stiffening mandrels, or for preferred dispensing geometry of the adhesive material. Nozzle 120 may include one or more hinged portions, such as to allow controlled bending prior to, during or after tissue fixation.

Nozzle 120 is fixedly or removably attached to housing 110 via attachment means 113, as have been described in detail in reference to FIG. 1. Nozzle 120 includes proximal portion 123 and distal portion 121, which includes at its distal end, tip 122. Tip 122 may be sharpened, such as to penetrate tissue such as soft tissue or bone, and may have an anti-coring tip such as a tip configured to avoid removing tissue cores during insertion. In a preferred embodiment, the anti-coring tip 122 includes a rigid tube construction with a bevelled edge. The distal end of the bevelled edge includes a sharpened tip end and the proximal end of the bevel edge includes a bead-blasted or otherwise buffed heal portion configured to avoid coring tissue. Tip 122 is configured to penetrate through the patient's tissue and any other material to be coapted to the patient's tissue. In an alternative embodiment, tip 122 has a blunt or otherwise atraumatic tip, such as to perform procedure at patient sites in which a sharpened tip may cause undesired damage to the patient, such as in the treatment of a patient burn.

Nozzle 120 is shown as a straight (linear) construction, preferably rigid but alternatively constructed to controllably transition from flexible to rigid or vice versa. In an alternative construction, nozzle 120 may comprise a curvilinear or other non-linear shape, such as a curvilinear shape configured to pass through tissue in a curvilinear manner, such as to avoid damaging certain internal body sites. Delivery device 100 includes housing 110 constructed in a pistol grip geometry with trigger 111 which is slidingly received by housing 110. Housing 110 includes cartridge 115. In one embodiment, cartridge 115 may be pre-loaded with adhesive and simply inserted into housing 110 prior to use. Multiple cartridges, such as cartridges including one or more similar or dissimilar adhesives may be included in system 10. In another embodiment, cartridge 115 is filled with a handheld fill device, not shown but typically a manual or automated fill device configured to inject one or more adhesives into cartridge 115. In another embodiment and as shown in FIG. 6, cartridge 115 is filled with adhesive via conduit 201 of base unit 200. Base unit 200 may be a table top or otherwise stationary piece of equipment, and includes a user interface with one or more controls and one or more display screens. Adhesive may be inserted into cartridge 115 prior to the clinical procedure, during the clinical procedure, or both, such as via a precision, pressure or volume controlled pumping system of unit 200. Conduit 201 may include one or more tubes or lumens through which adhesive travels into cartridge 115. Unit 200 may include a heating unit, such that the adhesive is heated to a temperature above room temperature or above body temperature. Unit 200 may include a power supply, such as a power supply connected to one or more components of device 100 through conduit 201. Unit 200 may provide a cooling feature, such as cooled saline or cryogenic fluid that travels to device 100 through conduit 201.

Housing 110 and trigger 111 are preferably connected to a mechanical control mechanism such as a mechanism including one or more levers, cams, linkages transducers, linear actuators, and/or other mechanical or electrical elements to allow trigger 111 to initiate the delivery of the adhesive material of the present invention. In one embodiment, the adhesive is in a flowable state at room temperature and trigger 111 initiates a pressurized delivery of the adhesive such as via a pressurized vessel (e.g. internal to housing, not shown but in fluid communication with cartridge 115 of housing 110. or a pressurization source of unit 200 connected to housing 110 via conduit 201.

In an alternative embodiment, delivery device 100 and the various flowable material delivery devices of the present invention, include a power supply such as a battery and electronics used to operably control one or more mechanisms of delivery device 100, and/or to deliver energy to produce heating used to produce an elevated temperature to make the adhesive material flowable. Activation of delivery device 100 may be manual, such as via linkages and other controls integral to device 100, or automatic or semi-automatic, such as via a control that activates a circuit controlling an electromechanical assembly or system such as an assembly or system including a motor, solenoid, or a piezo crystal.

In yet another alternative embodiment, the adhesive delivery system is provided in a kit form, including two or more delivery devices, nozzles, and/or adhesive materials. The two or more components may be similar, or may have different features. In a preferred embodiment, a kit includes two or more nozzles with different delivery characteristics. In another preferred embodiment, a kit includes two or more adhesive materials, such as adhesive materials with different melt temperatures; different thermal behaviors; different bioabsorption rates; different viscosities; different hardening times; or combinations of these.

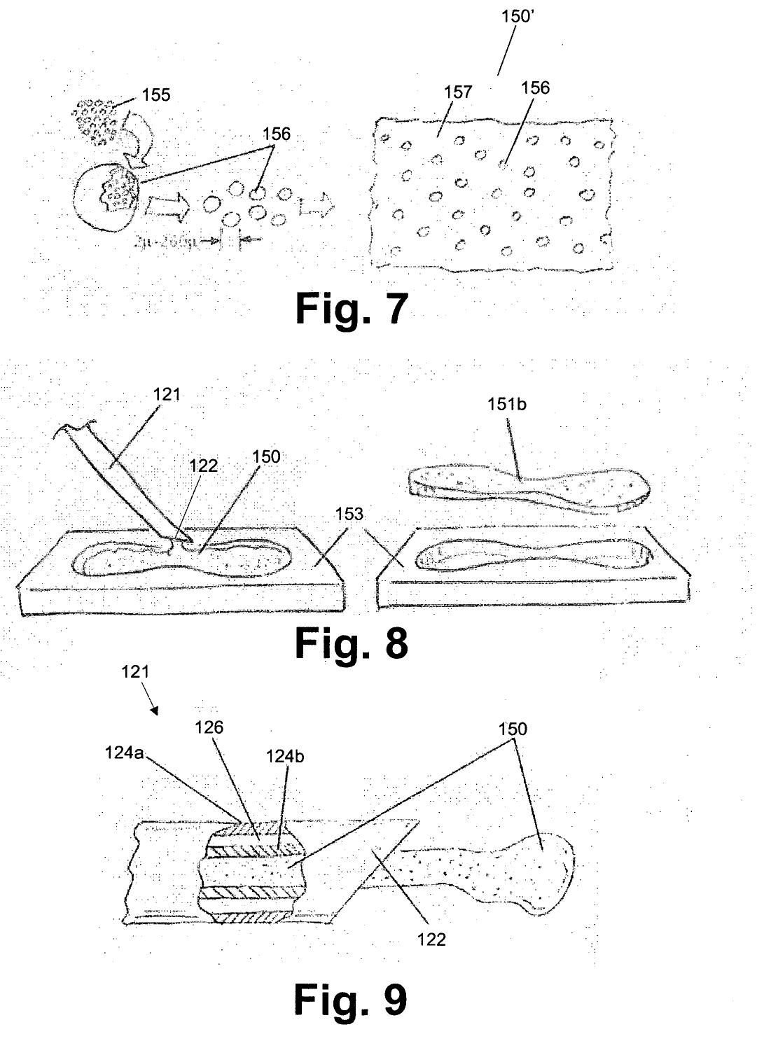

Referring now to FIG. 7, an adhesive material of the present invention is illustrated. Adhesive material 150 includes multiple capsules 156 containing microencapsulated treatment particles 155. Microencapsulation is a well-known method in the drug manufacturing field. The microencapsulated units range in a preferred state size from 2 microns to 260 microns. The capsules 156 would be formulated of a biocompatible material that could be of differing melt temperature, dissolution and/or flowable state characteristic than the base adhesive material 157. In one embodiment, adhesive material 157 may be made of bioabsorbable material and after being in place in the tissue, capsules 156 are released into the surrounding tissue to produce desired therapeutic results.

Referring now to FIG. 8, a perspective view of a system and method of manufacturing an implant is illustrated. The distal portion 121 of a device of the present invention is shown with tip 122 in close proximity to a cavity of fixture 153. Material 150 is flowing out of tip 122 into the cavity such as to form an implant with the shape of the cavity of fixture 153. Adhesive 150 is configured to harden or otherwise become continuously attached in the rigid, semi-rigid or flexible matrix 151b shown. Matrix 151b is a suitable implant to be introduced to a tissue repair site of a patient, and may be fixedly attached or otherwise secured using the adhesive of the present invention. Fixture 153 can be configured with one or more cavities, said cavities configured to produce a matrix in one or more forms. In a preferred embodiment, the cavity is configured to produce a matrix in a form selected from the group consisting of: a tube; a plate such as a flat plate; a pin such as a round pin; a filamentous structure such as a thread-like structure; a ribbon-like structure; a corrugated structure; a perforated structure; and combinations of these.

Referring now to FIG. 9, a side, partial sectional view of the distal portion of the delivery device of the present invention is illustrated. Distal portion 121 is shown with a multiple, coaxial tube construction including outer tube 124a and inner tube 124b separated by insulator 126. Insulator 126 may comprises an air gap, or one or more insulating materials. The outer surface of inner tube 124b and the inner surface of outer tube 124a may include a reflective surface such as to creating an insulating, thermos effect. Shown flowing through inner tube 124b and out of tip 122 is adhesive material 150, such as a material which has been heated to a sufficient temperature, such as a temperature slightly above body temperature, to cause adhesive material 150 to flow at a sufficient rate to perform a clinical procedure. Material 150 may be heated by a separate unit as described in reference to FIG. 6, or by an internal heating assembly as described in reference to FIG. 1. Alternatively or additionally, one or more heat shields can be included surrounding distal portion 121 such as to prevent undesired tissue damage from the heated adhesive material 150. Alternatively or additionally, a cooling element can be included such as a peltier element, a cryogenic element, a heat sink, or a cooled member such as a member cooled with cold saline.

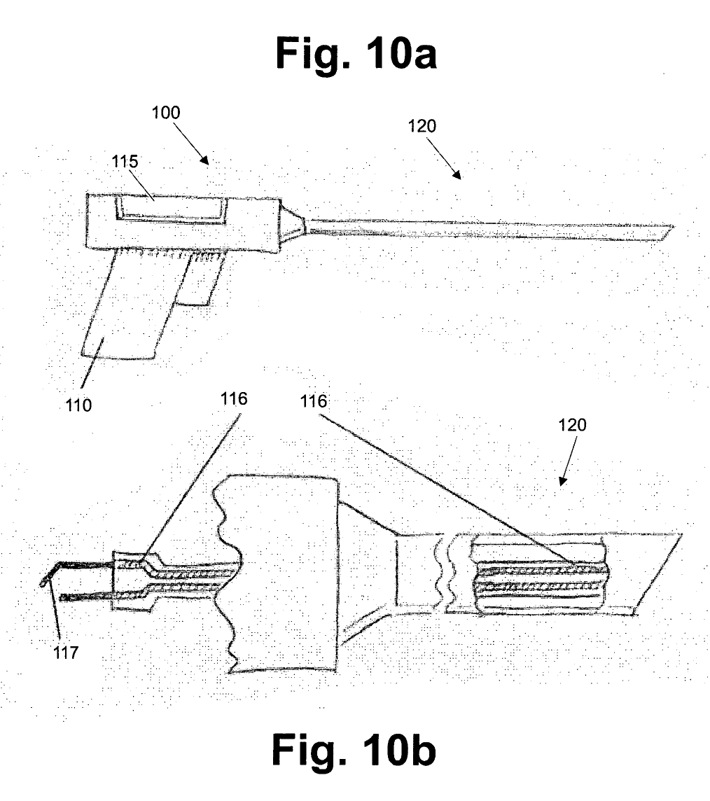

Referring now to FIGS. 10a and 10b, a side view and a side partial sectional view, respectively, of the delivery device of the present invention is illustrated. Delivery device 100 includes nozzle 120 which is attached to housing 110, housing 110 include cartridge 115, each of which have been described in detail in reference to multiple figures here above. Device 100 includes heating unit 116 which travels from housing 110 and into a lumen of nozzle 120 as shown in FIG. 10b, such as to heat the material within nozzle 120 as well as the material in housing 110. At the proximal end of heating unit 116 is switch 117, preferably connected to a trigger, not shown but preferably a trigger of housing 110 as has been described in detail here above.

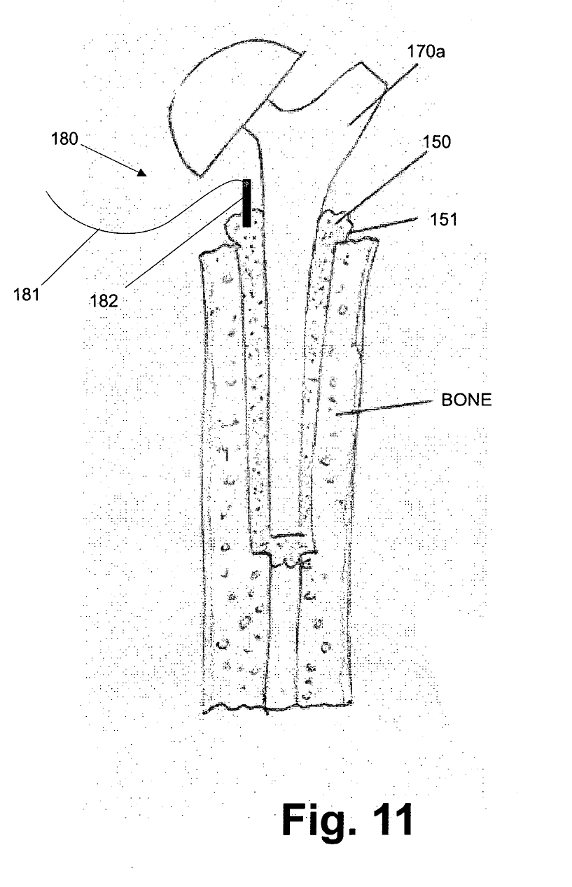

Referring now to FIG. 11, a side sectional view of the adhesive material and implant of the present invention is illustrated. Implant 113, a hip prosthesis, is shown fixated into the bone with adhesive materials 150, still in a flowable or malleable state, and adhesive material 151, which has transitioned to a hardened state. Alternative or additional to a hip prosthesis, the system and devices of the present invention can be used to secure numerous implantable prostheses including but not limited to: knee; shoulder; ankle; vertebral segments; elbow; metatarsal and metacarpal prosthesis.

Adhesive material 150, while in its softened state, can be reshaped by the clinician, such as with an insulated tool. Reshaping of adhesive material 150 can provide numerous benefits including providing a more secure implantation of implant 170a, and providing a smooth surface at the implant exit. In the embodiment of FIG. 11, a heating assembly 180 is provided to maintain adhesive material 150 in a malleable condition through heating. Heating assembly 180 includes heating element 182, shown placed into adhesive 150, and cable 181 a single or multi-conductor cable. Cable 181 is attached to a heat controller or simple power supply, neither shown but preferably a closed-loop unit based on measured temperature. Heating assembly 180 may be a resistive load, and may include a temperature sensor such as a thermocouple or thermistor, transmitting one or more signals through cable 181, and used to maintain heating assembly 180 at a specific temperature or temperature range (e.g. through control of electrical power delivered to heating assembly 180). In one embodiment, a self-heating thermistor is used that both provides a temperature signal as well as generates heat as power is supplied to it. Alternatively or additionally, one or more agents may be added to adhesive material 150 to prolong the conversion of flowable adhesive 150 to hardened material 151.

Systems of the present invention may include one or more energy sources to move the adhesive material from the ex-vivo housing to the in-vivo application site. Heating of the adhesive material may be required and one or more energy sources may be part of the present invention. Energy sources may also be used to activate mechanisms, position one or more components or assemblies of the delivery device, activate a cutoff mechanism or a mechanism used to make penetrating holes in tissue for better dispersion of the adhesive material, or for other purposes requiring energy.

Numerous kit configurations are also to be considered within the scope of this application. A transcutaneous tissue adhesion system is provided with one or more types of nozzles or preshaped casting kits to shape the material in preferred fashion to fit the repair site.

In yet another preferred embodiment, the adhesive material may be non-absorbable. The flow of the material may require heating to make the material achieve a flowable state, or the material may be configured to flow at room temperature. The material, once placed into a body, would stay there indefinitely.

In still yet another preferred embodiment, the adhesive material or matrix may contain hollow bodies that contain a biologically inert gas, liquid or solid in the widest possible range of mass fraction. The hollow bodies may be themselves absorbable or partially absorbable. They may be made from adhesive that melts at a different temperature than the bulk of the adhesive matrix. The adhesive matrix may be absorbable while the hollow bodies are non-absorbable. Or, conversely, the adhesive matrix may be non-absorbable while the hollow bodies are absorbable. Both the matrix and bodies may be absorbable or non-absorbable. They may be absorbable at the same or differing rates within the body.

In even yet another preferred embodiment, the material may be comprised of a mixture of adhesive matrix and adhesive bodies, and the bodies may have one or a multitude of contents. The bodies may be a mixture of bodies containing one material or several materials, such as gases, liquids or solids, absorbable materials or drugs or non-absorbable materials.

In a further preferred embodiment, the hollow bodies mentioned above may be comprised of polymer that melts at a temperature higher than the matrix polymer. Furthermore, there may be several types of bodies that possess a multitude of melting temperatures. The mixture may allow melting of the bodies, contained within the matrix, by raising the matrix temperature above its melting temperature to the melting temperature of the hollow body material or one of the constituent hollow body materials. This may have the intended effect of yielding a new blended polymer with resulting properties distinct from the matrix or spheres. These new properties may include, but are not limited to, melting temperature, non-absorbability, absorbability, chemical reactivity, color, elastic modulus, electrical conductivity, density, and porosity.

In yet a further preferred embodiment, that material above may contain some other geometric shapes, instead of hollow bodies. The shapes may be spheres, cylinders, strings, fibers, flakes, sheets, or random particles.

In yet a further preferred embodiment, the adhesive material matrix comprises a mixture of polymer and organic and/or inorganic compounds such that the organic and/or inorganic compounds are delivered due to the flowability of the base polymer.

In yet a further preferred embodiment, the adhesive material matrix comprises a mixture of polymer and organic and/or inorganic compounds such that the organic and/or inorganic compounds are processed due to processability of the base polymer. The processing may or may not require heat for the base polymer to flow.

In yet a further preferred embodiment, the adhesive material matrix comprises a mixture of polymer and organic and/or inorganic compounds such that the organic and/or inorganic polymer has a therapeutic value and the base polymer acts as carrier of that compound.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. In addition, where this application has listed the steps of a method or procedure in a specific order, it may be possible, or even expedient in certain circumstances, to change the order in which some steps are performed, and it is intended that the particular steps of the method or procedure claim set forth here below not be construed as being order-specific unless such order specificity is expressly stated in the claim.

1. An adhesive material injection system for delivering adhesive to a patient site comprising:
adhesive material; and
a delivery device comprising:
a housing; and
a nozzle, said nozzle comprising a proximal end and a distal end, wherein the adhesive material is configured to exit said nozzle distal end and wherein the housing comprises a reservoir for containing at least a first portion of the adhesive material.

2-47. (canceled)

48. The system of claim 1, wherein the reservoir is removably attached to the housing.

49-107. (canceled)

108. The system of claim 1, wherein the adhesive material is configured to be formed into one or more geometric shapes after exiting the nozzle.

109. The system of claim 108, wherein the adhesive material is formed into the geometric shape in-situ.

110. The system of claim 108, wherein the geometric shape is selected from the group consisting of: a tube; a plate such as a flat plate; a pin such as a round pin; a filamentous structure such as a thread-like structure; a ribbon-like structure; a corrugated structure; a perforated structure; and combinations thereof.

111-113. (canceled)

114. The system of claim 1, further comprising a second adhesive material, wherein the first adhesive material and the second adhesive material have a different characteristic selected from the group consisting of: melt temperature; bioabsorption rate; viscosity; hardening times; and combinations thereof.

115-134. (canceled)

135. An adhesive material injection system for delivering adhesive to a patient site comprising:
adhesive material; and
a delivery device comprising
a housing;
a nozzle, said nozzle comprising a proximal end and a distal end, wherein the adhesive material is configured to exit said nozzle distal end; and
a heating unit configured to maintain the adhesive material in a non-hardened state after said adhesive material exits the nozzle, wherein the heating unit comprises a temperature sensor.

136. The system of claim 135, wherein the heating unit comprises a heating element.

137. The system of claim 136, wherein the heating element is configured to be inserted into the adhesive material after the adhesive material has exited the nozzle.

138-140. (canceled)

141. The system of claim 1, further comprising an anchor device.

142. The system of claim 141, wherein the anchor has a filamentous structure.

143. The system of claim 142, wherein the anchor comprises a loop on one end.

144-145. (canceled)

146. A method for treating a patient, said method comprising: providing the adhesive material injection system of claim 1, applying said system to a patient to repair a first portion of soft tissue and a second portion of soft tissue different to said first portion.

147-155. (canceled)

156. A method for treating a patient, said method comprising: providing the adhesive material injection system of claim 1 and applying the system to a patient to repair defects in the nasal cavity.

157-160. (canceled)

161. A method for treating a patient, said method comprising: providing the adhesive material injection system of claim 1 and applying the system to a patient to apply the bone adhesion material to implantable prosthesis.

162-174. (canceled)

175. A method of for treating a patient, said method comprising: providing the adhesive material injection system of claim 1 and dispensing the adhesive material below the skin surface of the patient.

176-193. (canceled)

194. A method for treating a patient, said method comprising: providing the adhesive material injection system of claim 1, using the adhesive material injection system to dispense adhesive material, and implanting an anchoring device.

195-205. (canceled)

206. The system of claim 1, wherein the adhesive material is non-bioabsorbable and comprises hollow bodies and a matrix material, wherein the hollow bodies comprise at least one polymer having a melting temperature or thermal behavior that is different from that of the matrix material.

207-209. (canceled)

210. The system of claim 1, wherein the adhesive material is non-bioabsorbable and comprises a matrix and at least one of spheres, fibers, flakes, cylinders, or randomly sized and shaped particles.

211. The system of claim 210, wherein the adhesive material comprises at least one of spheres, fibers, flakes, cylinders, or randomly sized and shaped particles that are comprised of a polymer having a melting temperature or thermal behavior that is different from that of the matrix material.

212-241. (canceled)
